(19) United States
(12) Patent Application Publication
(10) Pub. No.: US 2016/0199967 A1
(43) Pub. Date: Jul. 14, 2016

(54) FASTENER HEAD AND COMPLEMENTARY DRIVER

(71) Applicant: Guido Stahl, Toronto, CA (US)

(72) Inventor: Guido Stahl, Toronto, CA (US)

(21) Appl. No.: 14/778,367

(22) PCT Filed: Oct. 18, 2013
(86) PCT No.: PCT/CA2013/000890

§ 371 Date: Sep. 18, 2015

(30) Foreign Application Priority Data
Mar. 20, 2013 (CA) 2809884

(51) Int. Cl. B25B 23/12; A61B 17/88; A61B 17/86; F16B 23/00; B25B 15/00

(52) U.S. Cl. CPC B25B 23/12; F16B 23/0023; B25B 15/005; A61B 17/8615; A61B 17/8888

(57) ABSTRACT

A fastening system comprising a driver tool bit and a complementary fastener head is provided. The coupling between the driving end of the driver tool bit and the fastener head occurs via at least one protruding ledge located on the driving end, and a cavity in the fastener head shaped and sized to receive the ledge upon a rotation of the vertical Y axis (e.g. lengthwise or along a longitudinal axis) of the inserted driving end. More particularly, the cavity of the fastener incorporates an overhang which is configured to mate with the protruding ledge of the driving end of the driver tool bit, resulting in an enhanced connection during insertion or removal of the fastener from a work piece, without requiring the application of longitudinal axis pressure directed towards the work piece.

### FIELD OF THE INVENTION

The present invention relates to the field of tool bits and fasteners with complementary receiving heads.

### BACKGROUND OF THE INVENTION

There are many different types of driver tool bits and complementary fasteners known in the art. However, the existing designs require the application of a certain amount of longitudinal axis pressure (i.e. along the vertical Y axis, the pressure directed towards the work object in which the fastener is being or has been inserted) to insert or remove a fastener. For example, referring to U.S. Pat. Nos. 5,203,657, 5,205,694 and 5,890,860, the wallboard screw with bit tapered screw driver tip requires the application of vertical axis pressure to keep the tapered driver bit in the socket head of the wallboard screw while inserting into a work object.

In other words, vertical axis pressure directed towards the work object is required to be applied in order to maintain the connection between the driver bit and the socket head of the screw, even when removing the screw from the work object. Particularly when screws have become damaged, the requirement to maintain this vertical axis pressure towards the work object, when it is ultimately desired to move the screw in an opposite direction away from the work object, becomes awkward. It can also lead to further damage to the screw and work object, which can happen in a number of ways. For instance, fastener heads may be damaged due to their slipping while coupled to driver tool bits. To compensate for the slippage effect, vertical axis pressure is applied. This can cause damage to the integrity of the driver tool bit or, more commonly, to the fastener head being installed. For example, due to such pressure, the fastener head of a screw or bolt may become “stripped”, meaning that the aperture in the fastener head, which is shaped to mate with the end of the driver tool bit, has lost its shape due to shearing caused by the force of the driver tool bit end combined with the downward longitudinal axis pressure. Such stripped screws or bolts become very difficult to remove.

Likewise, during the removal of a fastener from a work object, a slight reverse axial force (i.e. in a direction away from the work object) will result in the driver tool bit disengaging from the fastener. Repeatedly inserting and applying vertical axial force to the head of the fastener to prevent this disengagement can result in additional damage to the grooved recesses of the head of the fastener. Such damage to the fastener head can also cause undue stress and pressure on the work object receiving the fastener, which can result in the aperture receiving the fastener becoming damaged itself.

Furthermore, having to apply vertical axial force towards the work object to install screw type fasteners of the type currently on the market could make it more difficult to utilize these tools in applications and environments that are less typical. For instance, in an underwater or outer space environment in which the user's positioning is less secure in relation to the work surface, the application of vertical axial force towards the work object would result in an equivalent force being generated which pushes the user away from his or her task. In such an environment it would be advantageous to have a more secure connection between tool bit and fastener, which does not require the application of vertical axial force towards the work object in order to maintain the connection.

There are other applications in which it is particularly desirable to avoid unnecessary damage to the work object itself. Such applications include any which involve expensive or easily marred work surfaces, or delicate surfaces such as tissues or bone encountered in medical and surgical applications.

Accordingly, there is a need for a non-slip connecting driver tool bit and complementary fastener that will minimize the aforementioned slipping and disengagement by allowing the tip of the driver tool bit and the complementary head of the fastener to remain physically coupled together during the installation and/or removal of a fastener from a work object, such as to minimize any additional vertical pressure that is required for their use.

### SUMMARY OF THE INVENTION

In one aspect, there is provided a driver tool bit for engaging a receiving cavity of a fastener head including an overhang located over the receiving cavity, the driver tool bit comprising a shank end and a driving end opposite the shank end, the driving end comprising at least one blade and one or a plurality of protrusions incorporating ledges, each protrusion located on one of said blades; wherein once the driver tool bit rotatably engages the fastener head the protrusions are received within the cavity and located at an underside of the overhang. Thus, the physical abutment and contact of each protrusion against the overhang inhibits the tool bit from disengaging from the fastener during use. Preferably, each protrusion is formed by at least a ledge extending transversely to the longitudinal axis of the driver tool bit, the ledge positioned and sized to be received into the receiving cavity of the fastener head and to cause physical abutment with the overhang, when the driver tool bit is rotated relative to the fastener head.

In a further aspect, there is provided a fastener head for engaging a driver tool bit having a driving end that comprises at least one blade and at least one protrusion with a ledge, the protrusion located on one of said blades. In order to provide the mating connection, the fastener head comprises a receiving cavity with at least one overhang located over it, the overhang configured to engage with the ledge of the driving end of the driver tool bit.

In a further aspect, there is provided a fastening system comprising a driver tool bit and complementary fastener head, wherein the driver tool bit comprises a shank end and a driving end, the driving end comprising at least one blade and one or a plurality of protruding ledges, and wherein the complementary fastener head comprises a receiving cavity including an overhang located over it, the overhang configured to mate with the ledge when the driving end is inserted and rotated. Thus the result is an enhanced connection between driver tool bit and fastener, which does not require the application of additional vertical axis pressure to keep them together in use, and which reduces the likelihood that the components or the work surface will become damaged in use.

### BRIEF DESCRIPTIONS OF DRAWINGS

The invention will be now be described in further detail with reference to the following figures and exemplary embodiments.

FIG. 1A is a side elevation view of a driver tool bit configured for connection with a complementary fastener (example shown in FIGS. 2A-2C);

FIG. 1B illustrates a perspective view of the driver tool bit;

FIG. 1C illustrates a second side view of the driver tool bit, opposite to the side shown in FIG. 1A;

FIG. 1D illustrates an end elevation view, taken along section lines A-A of FIG. 1A, of the driver tool bit and its blade arrangement according to one embodiment, the view being oriented towards the shank (2) which is attached to the shank end (4) of the driver tool bit;

FIG. 1E illustrates an end elevation view of the driver tool bit, also taken along section lines A-A of FIG. 1A, the view being oriented towards the tip (1) of the driver tool bit;

FIG. 2A illustrates a top elevation view of an exemplary fastener with a flat head configured to engage the driver tool bit depicted in FIGS. 1A-1E;

FIG. 2B illustrates a cross-sectional side view of the fastener of FIG. 2A along lines B-B;

FIG. 2C illustrates a side view of the fastener of FIGS. 2A and 2B;

FIG. 3A illustrates a top view of the fastener head of FIGS. 2A-2C;

FIG. 3B illustrates an isometric view of the fastener head of FIGS. 2A-2C;

FIG. 3C illustrates a side view of the fastener head of FIGS. 2A-2C;

FIG. 4A illustrates a portion of the fastener head of FIGS. 2A-2C and a portion of the driver tool bit of FIG. 1A prior to engagement;

FIG. 4B illustrates the fastener head and the driver tool bit of FIG. 4A during engagement;

FIG. 4C illustrates a cross-sectional view of the driver bit inserted into the fastener head;

FIG. 4D illustrates a cross-sectional view of the driver tool bit as seen in FIG. 4B inserted into the fastener head and rotated in a clockwise position;

FIG. 4E illustrates a cross-sectional view of the driver tool bit as seen in FIG. 4B inserted into the fastener head and rotated in a counter-clockwise position;

FIG. 5A illustrates a side view of the fastener and the driver tool bit prior to engagement;

FIG. 5B illustrates a side view of the fastener and the driver tool bit during engagement;

FIG. 6A illustrates a top elevation view of a manufactured precursor for the fastener head prior to being stamped to achieve its final form;

FIG. 6B illustrates a cross-sectional view of the precursor of FIG. 6A, taken along lines C-C;

FIG. 6C illustrates an isometric view of the precursor;

FIG. 6D illustrates a side view of the precursor;

FIG. 7A illustrates a further side view of the fastener head as previously seen in FIGS. 3A-3C.

FIG. 7B illustrates a cross-sectional view of the fastener head of FIG. 7A, taken along lines D-D.

### DETAILED DESCRIPTION OF THE DRAWINGS

In one aspect of the present invention, there is provided a fastening system that comprises a driver tool bit and a complementary fastener head that allows physical coupling and direct abutment therebetween. Preferably, the coupling occurs via at least one protruding ledge located at the driving end of the driver tool bit and a fastener head having a surface with a cavity shaped and sized to receive and secure the protruding ledge upon a rotation about the vertical Y axis (e.g. lengthwise or along a longitudinal axis) of the inserted driver tool bit. In a preferred embodiment, minimal longitudinal axis pressure directed towards the work object (i.e. the axis along the length of the driver tool bit) is required to insert or remove a fastener from a work object.

In another aspect, there is provided a driver tool bit configured for connecting with a complementary fastener head with a receiving cavity. The receiving cavity can be formed into the top of a fastener such as a screw or machine bolt, which may be designed for use in securing wood, metal and composite materials. The fastener itself may be made from a variety of materials including metal, ceramic, or plastic.

In a further aspect, the driver tool bit is manufactured such that small ledges or protrusions on its driving end surface are capable of removably coupling with a complementary receiving fastener having an overhang over its receiving cavity, resulting in a secure connection during insertion or removal from a work piece, without requiring additional longitudinal axis pressure along the length of the tool bit driver (e.g. directed towards the work object). In this manner, when the driver tool bit engages the fastener, the ledges located at the driving end of the tool bit are received at an underside of the overhang and prevent the tool bit from slipping relative to the fastener. The connecting driver tool bit can be magnetic to facilitate certain applications. The integrity of the connection of the tool bit and fastener head enhance the functional life of both the tool bit and the complementary fasteners, as well as reducing the likelihood of damage to the work object.

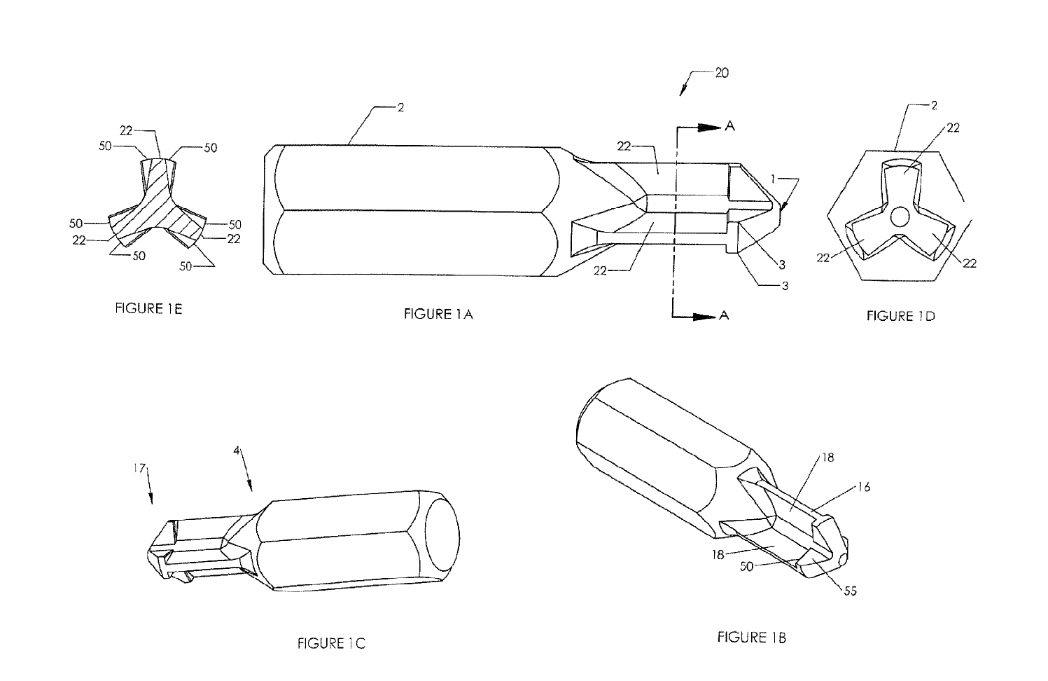

Referring to FIG. 1A, shown is a side view of a driver tool bit 20 attached to a shank 2, the driver tool bit 20 comprising at least one blade 22 at its driving end 17 (seen in FIG. 1C), the driving end 17 terminating at a tip 1. As seen in FIG. 1C, the driver tool bit 20 further comprises a shank end 4 (opposite to the tip end 1) that can be made or formed as a generic tool bit shank, which may be designed to be reversibly or permanently attached to a handle or power tool, as envisaged by a person skilled in the art.

In FIG. 1A there is shown an embodiment having three blades 22 (only two blades 22 being visible in this view) whereby each blade 22 has at least one protrusion 3 formed thereon. In the embodiment illustrated, as best seen in FIG. 1B, each blade consists of at least two faces 18 and one edge 16, each face 18 meeting a corresponding face 18 of another blade 22 at predefined angle (e.g. 120°). In the embodiment illustrated, three blades 22 are present, radiating from the axial center defined by tip 1, each blade having two protrusions 3. In an alternate embodiment, a single blade driver may be used and a protrusion 3 may be formed on at least one surface of the blade 22.

Each protrusion 3 can be made of metal or any other material known to driver tool bits. In a preferred embodiment, and as best seen in FIG. 1B, each protrusion 3 is further defined by at least two surfaces, a ledge 50 and an inner plane 55. The ledge 50 faces opposite tip 1 and extends transversely along the longitudinal axis of driver tool bit 20. The inner plane 55 extends longitudinally towards tip 1, and is perpendicular to ledge 50. In one embodiment, each protrusion 3 can be shaped of a generally triangular shape with the apex closest to (e.g. adjacent to) the tip end 1 and the base of the triangle located distant or away from the tip end 1. In an alternative embodiment, the protrusions 3 can be shaped as any polygon with three or more sides which are line segments and with at least two vertices distant (e.g. the base of the triangular polygon shape) from tip 1.

Each protrusion 3 incorporates the ledge 50 that is perpendicular to and adjacent to both face 18 and inner plane 55. In one preferred embodiment, the ledge 50 is formed of a rectangular shape extending outwardly from the longitudinal axis of driving end 17, however, in an alternate embodiment, the ledge 50 can be shaped in other polygon configurations.

Referring now to FIGS. 1D and 1E, respectively illustrated are two end elevation views of a three blade configuration of the driver tool bit 20 of FIG. 1A. Specifically, FIG. 1E illustrates the cross-sectional view of the driver tool bit 20 of FIG. 1A along lines A-A, the view being directed towards tip 1. In FIG. 1D, the view is directed in an opposite direction towards shank 2.

In FIG. 1E, there are two protrusions 3 on each blade, each protrusion 3 incorporating a ledge 50. Since each blade 22 incorporates two protrusions 3, and each protrusion 3 incorporates a ledge 50, a total of six ledges 50 are visible in this cross-sectional view.

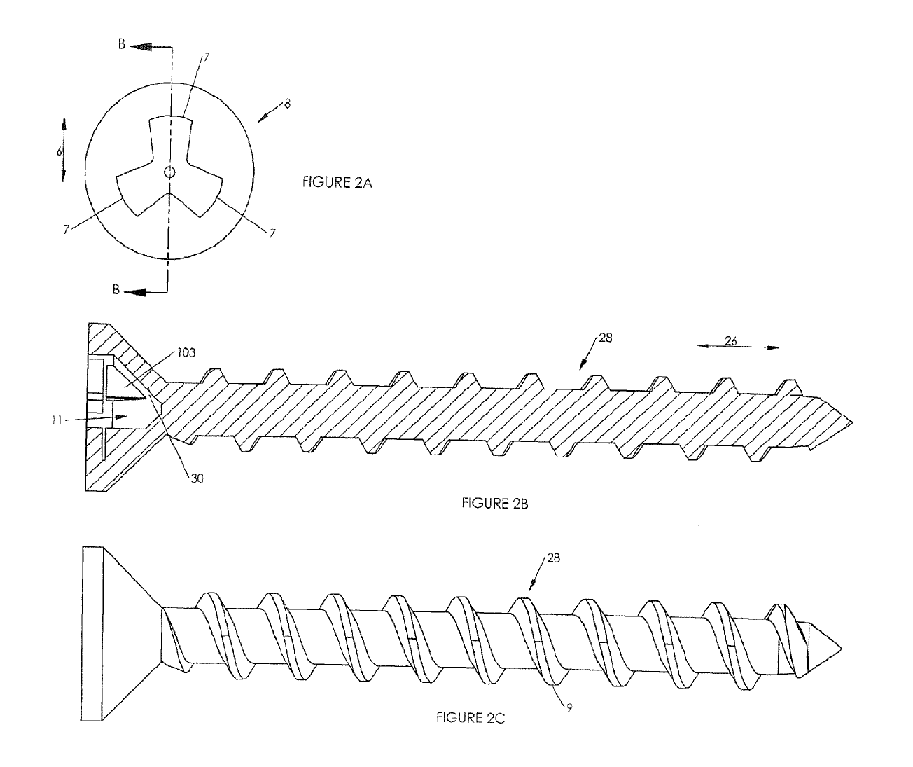

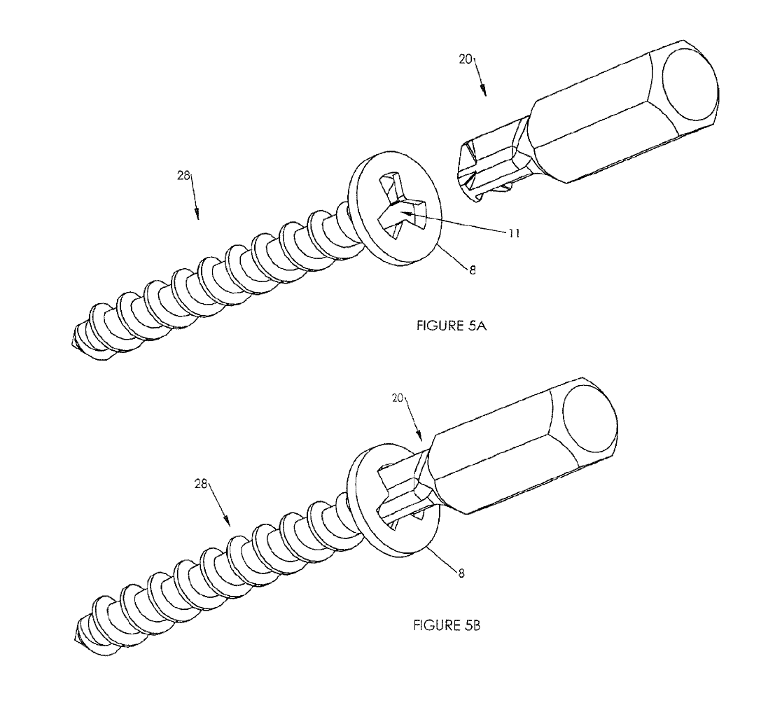

The driver tool bit 20 is designed to mate with a complementary fastener. The relationship is generally depicted in FIGS. 5A and 5B. Specifically, tip 1 is received into a complementary receiving cavity 11 that is cut into fastener head 8 of fastener 28. Further details of the fastener 28 are shown in FIGS. 2A-2C. FIG. 2A displays a top elevation view of a fastener head 8 having three slots 7 defining the surface of a receiving cavity 11. FIG. 2B illustrates a cross-sectional side view of the fastener of FIG. 2A along lines B-B. Receiving cavity 11 is configured to accept at least a portion of the driver tool bit tip 1. As illustrated, the fastener 28 is shown as a flat head screw fastener but other shapes of the fastener head 8 could be envisaged. FIG. 2C further depicts a side view of the fastener 28 and the cylindrical portion of the fastener 28 extending from the underside of the fastener head 8 to the tip is defined as a threaded shank 9 having a winding thread, which can be varied as envisaged by a person skilled in the art.

There is also shown a longitudinal axis 26 which is also referred to as a turning axis by which the fastener 28 is turned by the driver tool bit 20 of FIGS. 1A-1C. The transverse axis 6 which extends along the surface of fastener head 8 is also shown.

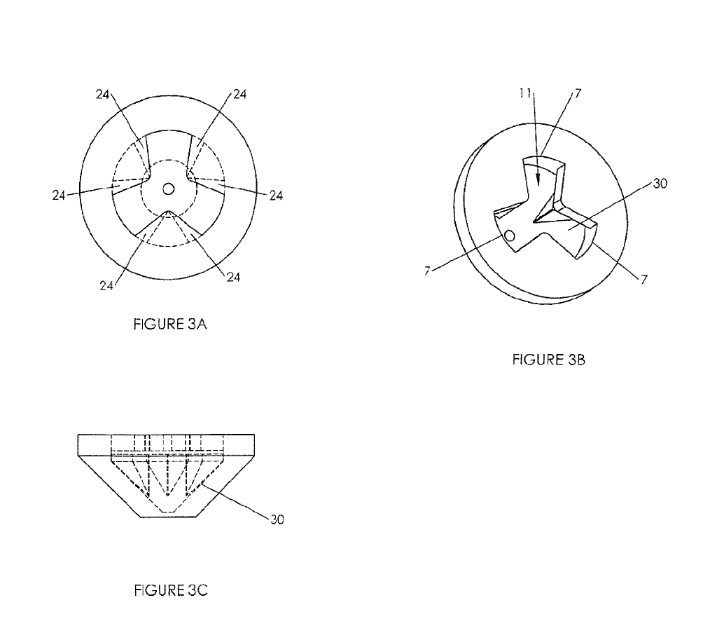

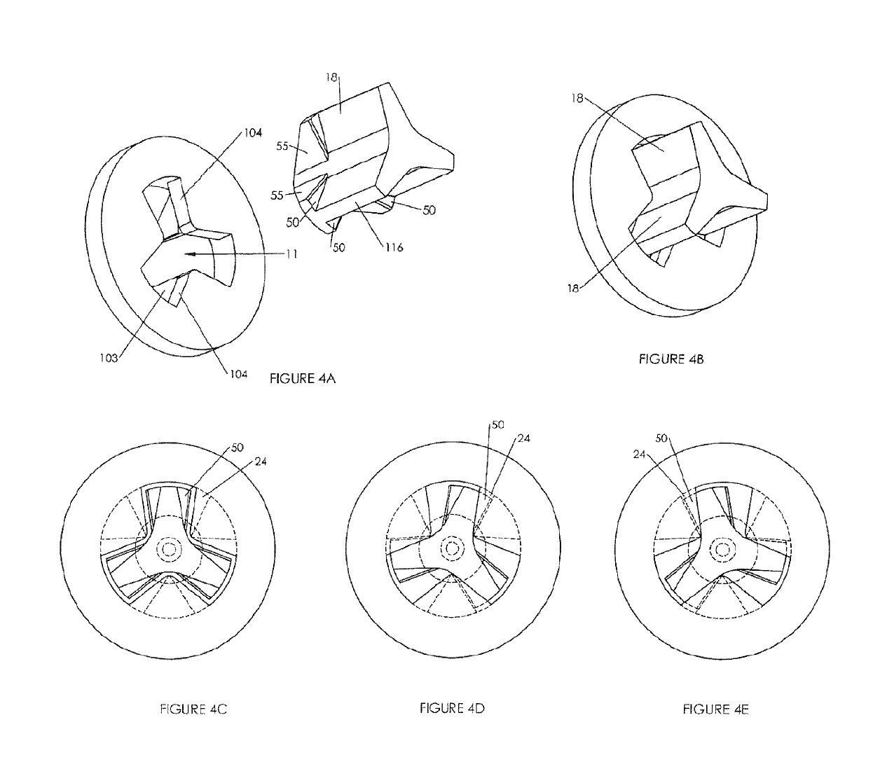

As further detailed in FIGS. 3A-4E, driver tool bit 20 is adapted for being received within a receiving cavity formed in the head of a screw or other fastener 28. Typically, the receiving cavity 11 can include one or more slots to fit the tip end 1 of the driver tool bit 20 (e.g. a screwdriver, wrench or drill driver bit). Depending on the number of slots, the shape of the receiving cavity may be a slit, a Y-shape as seen in the figures, cross, or other shape.

Referring to FIGS. 3A and 4A, overhangs 24 present in the receiving cavity 11 of fastener head 8 cooperate to receive the one or more protrusions 3 located at tip 1 of driver tool bit 20. Specifically, ledges 50 located on the underside of protrusions 3 contact overhangs 24 such as to securely engage the fastener 28 to the driver tool bit 20 upon a clockwise or counter-clockwise rotation about the turning axis 26 of the driver tool bit 20. The driver tool bit 20 is thereby prevented from slipping by the engagement of its one or more protrusions 3 with corresponding overhangs 24 contained within the receiving cavity 11 of fastener head 8.

Referring to FIG. 3A, which illustrates a top view of a cross-section of the fastener head 8, overhangs 24 can be more readily seen. The overhangs 24 are preferably located at each of the inner vertices of the three slots defining the surface of receiving cavity 11 for receiving tip 1 of the driver tool bit 20 of FIG. 1A-1C in the fastening or removal operation. Overhangs 24 are located parallel to the transverse axis 6 and extend towards a center of the turning axis 26. Ledges 50 of the protrusions 3 are configured to engage with and contact the overhang 24 in order to prevent the protrusions 3 from inadvertently slipping out through the slots 7 of the fastener head 8.

Referring to FIGS. 4A-4E, shown is the engagement of tip 1 of driver tool bit 20 having a three blade configuration, and two protrusions 3 located on each of the blades 22. As shown, tip 1 is received into the fastener head 8 and its receiving cavity 11. The enhanced connection that results can be understood by comparing FIGS. 4C-4E. In FIG. 4C, there has been no rotation of tip 1, therefore ledge 50 does not hook under overhang 24. However, in FIG. 4D, shown is a view of the driver tool bit 20 and its tip 1 which has been inserted into the receiving cavity 11 of fastener head 8, then rotated in a clockwise direction. The protrusions 3 on each of the three blades 22 are secured within the receiving cavity 11 upon a rotation of the driver tool bit 20 about the turning axis 26 (see FIG. 2B). Accordingly, the secure mating of the protrusions 3 provides the engagement of the ledge 50 below the overhang 24. In this way, the coupling between the driver tool bit 20 to the fastener head 8 occurs such that longitudinal axis pressure is not needed to maintain the connection while inserting a fastener into a work object. In other words, the driver tool bit 20 is less likely to become inadvertently disengaged while being used to drive fastener 28 into a work object. Once received therein, the ledge 50 provided by the protrusions 3 cooperate with overhang 24 to prevent slippage of the mating connection between fastener head 8 and driver tool bit tip 1.

Referring now to FIG. 4E, shown is a top elevation view of a fastening system including a driver tool bit 20 having at least one blade 22 and inserted into a receiving cavity 11 of a fastener head 8 and rotated in a counter clock wise position. As illustrated, similarly as for FIG. 4D, the protrusions 3 are again secured and physically held below the overlap portion 24 positioned on the surface of the fastener 28. The result is that even if reverse longitudinal axis pressure is applied, the fastener head 8 and driver tool bit 20 will remain engaged as long as the protusions 3 and overlap portions 24 remain in contact. This is an advantageous feature of the invention, in that it therefore becomes possible to “pull back” on the fastener head 8 when engaged with the driver tool bit 20, which facilitates removal of the fastener 28. This feature becomes important particularly when fastener head 8 or threaded shank 9 of fastener 28 have become partially stripped or otherwise damaged, or when the aperture in the work object holding fastener 28 has become damaged. Any of these forms of damage tend to render fastener 28 difficult to remove. By utilizing the reverse longitudinal axis pressure made possible by the configuration of protusions 3 and overhangs 24, the process of easing out such a fastener becomes much facilitated.

Also visible in cross section of FIG. 3C are sidewalls 30 of the recessed cavity 11, which are also illustrated in FIG. 2B. Sidewalls 30 extend downwardly from the surface of the fastener head 8 towards the threaded shank 9 to define the recessed cavity 11 for receiving tip 1 of the driver tool bit 20 and its blades 22. The protrusions 3 are also received within the recessed cavity 11 and located adjacent to the sidewalls 30 during the fastening operation or unfastening operation (e.g. rotation of the fastener).

In addition to the contact between overhangs 24 and ledges 50, there are further points of contact. As seen in FIG. 4A, the inner plane 55 of protrusion 3 will directly contact surface 103. Face 18 of the blade 22 will directly contact edge 104 of slots 7. Tip 1 also contacts sidewalls 30. What results from these multiple points of contact is an improved distribution of forces and a stronger, more robust connection between the driver tool bit 20 and the fastener 28. These additional points of contact further minimize the likelihood that the driver tool bit 20 will inadvertently disengage fastener head 8. However, to deliberately disengage them from each other, it is simply a matter of rotating the driver tool bit 20 so as to disengage ledges 50 of protrusions 3 from overhangs 24.

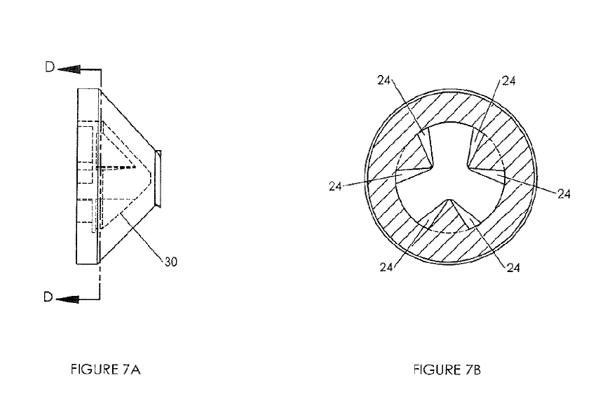

FIG. 7A illustrates a further side view of the fastener head 8, with the cross-section taken at line D-D seen in FIG. 7B. The overhangs 24 can be readily seen in FIG. 7B.

The recessed cavity 11 is a formed section within the fastener head 8 and is shown as a Y-shape, but is customizable. Other slot shapes can be envisaged such as a single slot shape having an overhang or protruding rim edge extending towards the center of the fastener head 8. Alternatively, four or five or more slots forming a cross or star shape are possible.

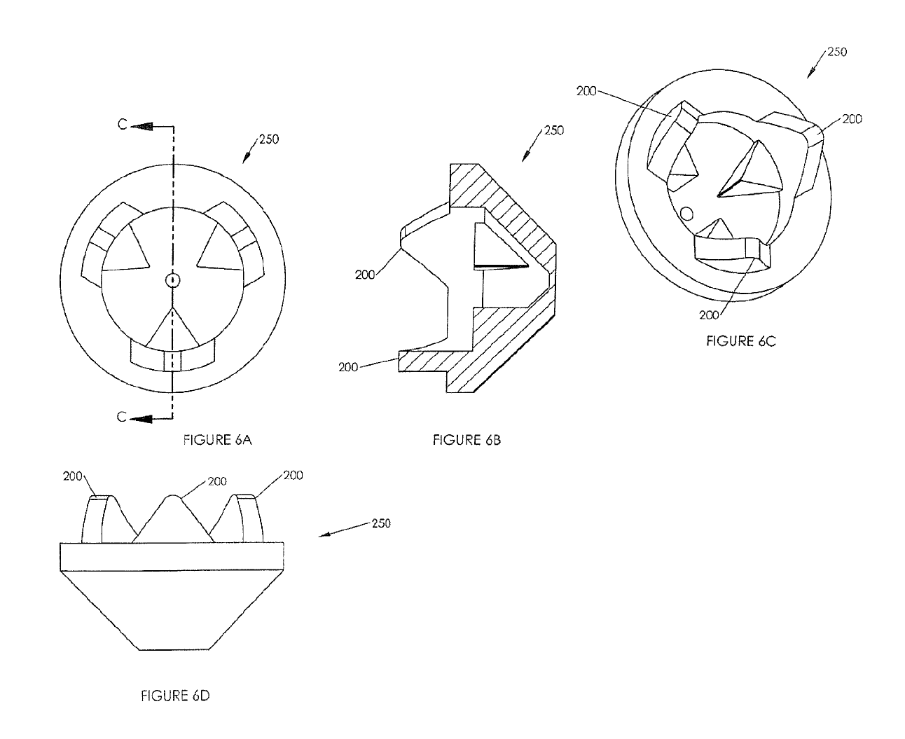

FIGS. 6A-6D illustrate a way of manufacturing a fastener such as that depicted in FIGS. 2A-2C, having a three-slot configuration for its receiving cavity. The manufacturing would typically take place in two steps. First, a precursor for the fastener head could be created by casting or by stamping the shape from a suitable metal or alloy. FIG. 6A is a top elevation view of a precursor 250, also depicted in FIGS. 6B-6D. Precursor 250 is formed as shown with three protruding Y-slit segments 200. The second step would be to stamp the Y-slit segments 200, in a direction 90 degrees downward and towards the centre of the head, to create the ledges 50 (which are most readily seen in FIG. 1E), and to form the top of the receiving cavity 11 seen, for example, in FIG. 2A.

Although what is depicted in the figures is a fastener screw having a head with a flat surface generally designed to be flush with the surface of a work piece following installation, as referred to previously there are many varieties of shapes known for fasteners. For example, the fastener head may have a rounded upper surface so that it protrudes from the work piece when installed. The fastener may or may not incorporate a threaded shank. Many variations are possible within the scope of this invention.

While the invention has been described with reference to specific embodiments, it will be appreciated that numerous variations, modifications, and embodiments would be evident to a person of skill in the art.

1. A fastener for receiving a driver tool bit having one or more protrusions at a driving end, the one or more protrusions tapering towards a tip of the driving end, the tip having a surface area oriented perpendicularly to the longitudinal axis of the driver tool bit, the one or more protrusions extending transversely to a longitudinal axis of the driver tool bit, the fastener comprising:
(a) a head having a receiving cavity and at least one slot defining a surface of said receiving cavity, for receiving the tip and the one or more protrusions; and
(b) at least one overhang located adjacent to the slot and extending transversely therefrom over the receiving cavity, the overhang configured for directly engaging and retaining one of said one or more protrusions within the cavity while the fastener is being fastened or unfastened,
and wherein the receiving cavity is further defined by a floor configured to mate with the tip of the driver tool bit, the floor having a surface area which substantially corresponds to the surface area of the tip of the driver tool bit;
and wherein the receiving cavity is further defined by sidewalk that extend between the floor and the one or more overhangs, said sidewalk circumferentially enclosing the floor and configured to mate with the one or more protrusions of the driver tool bit.

2. The fastener of claim 1 wherein there are two overhangs extending transversely from the at least one slot.

3. The fastener of claim 1 wherein there are three slots defining the surface of the receiving cavity, each slot having two overhangs extending transversely therefrom.

4. The fastener of claim 1 wherein at least one of said overhangs is configured to engage a protrusion of the driver tool bit upon insertion and rotation of the driver tool bit in a clockwise direction.

5. The fastener of claim 1 wherein at least one of said overhangs is configured to engage a protrusion of the driver tool bit upon insertion and rotation of the driver tool bit in a counter clockwise direction.

6. The fastener of claim 1, wherein there are three slots radiating outwards from the floor of the receiving cavity at angles of 120 degrees relative to each other.

7. A driver tool bit for engaging a receiving cavity of a fastener head, said receiving cavity including one or more slots, said slots being father defined by edges, and one or more overhangs over the receiving cavity, the receiving cavity being further defined by a floor, and sidewalls that extend between the floor and the one or more overhangs, said sidewalk circumferentially enclosing the floor;
the driver tool bit comprising:
(a) a shank end; and
(b) a driving end opposite the shank end, the driving end comprising one or more blades each having at least one surface and at least one protrusion located thereon, each blade arranged to radiate outwards from a tip of the driving end;
wherein once the driver tool bit rotatably engages the fastener head the one or more protrusions are received within the cavity and located under the one or more overhangs, the tip contacts the floor of the receiving cavity, and the surfaces of the one or more blades contact the edges of the one or more slots;
the engagement of the protrusions inhibiting the driver tool bit from disengaging from the fastener during rotation.

8. The driver tool bit of claim 7 wherein the one or more protrusions respectively comprise one or more ledges extending transversely from the driving end, the one or more ledges being configured to engage the one or more overhangs.

9. The driver tool bit of claim 7 wherein the one or more protrusions are triangle shaped, the apex of the triangle being located at the tip of the driving end.

10. The driver tool bit of claim 7 configured to connect at its shank end to manual or power driven driver devices.

11. The driver tool bit of claim 7 having three blades, each blade having two surfaces, each surface having a protrusion, the protrusions configured to engage with the overhangs contained within the receiving cavity of the fastener head.

12. The driver tool bit of claim 7 wherein the protrusions additionally comprise inner faces that are oriented perpendicular to the ledges, said inner faces configured to mate with side ails in the receiving cavity in the fastener head.

13. The driver tool bit of claim 7 wherein there are three blades arranged at angles of 120 degrees relative to each other.

14. The driver tool bit of claim 7 wherein each blade has two surfaces and two protrusions located thereon.

15. A fastening system comprising:
(a) a driver tool bit having a longitudinal axis and a driving end and a shank end, the driving end having a tip, the tip having a surface area oriented perpendicularly to the longitudinal axis of the driver tool bit, the driver tool bit comprising at least one blade, each blade having at least one protrusion extending, at a transverse axis therefrom and positioned at the driving end of the driver tool bit, each protrusion further comprising a ledge extending transversely to the longitudinal axis of the driver tool bit, each protrusion radiating outwards from the tip;
(b) a fastener head for receiving the driver tool bit, the fastener head comprising:
(i) a receiving cavity and at least one slot located at a surface of the fastener head, for receiving the tip of the driving end and said at least one blade and said at least one protrusion; and
(ii) an overhang located at the surface and adjacent to the slot and extending along the transverse axis such that the overhang and the surface form a cavity at an underside thereof, the overhang configured for engaging and retaining the at least one protrusion at its underside and within the cavity while the fastener head is being fastened or unfastened; and
(iii) the receiving cavity being further defined by a floor configured to mate with the tip of the driver tool bit, the floor having a surface area which substantially corresponds to the surface area of the tip of the driver tool hit; and
(iv) the receiving cavity being further defined by sidewalls that extend between the floor and the overhang, said sidewalls circumferentially enclosing the floor and configured to mate with the protrusions of the driver tool bit.

16. The fastening system of claim 15, wherein the driver tool bit inserted into the fastener head securely engages therewith upon one of a clockwise or counter clockwise rotation about the longitudinal axis of the driver tool bit with respect to the fastener head.

17. The fastening system of claim 15, wherein the driver tool bit and fastener head are magnetic.

18. The fastening system of claim 15, wherein the driver tool bit comprises three blades, each blade having two surfaces, each surface having one protrusion extending transversely therefrom, and the receiving cavity of the fastener head is defined at the surface by three slots radiating outwards from the floor of the receiving cavity at angles of 120 degrees relative to each other.

19. The fastening system of claim 15, wherein the protrusions additionally comprise inner faces that are oriented perpendicular to the ledges, said inner faces configured to mate with the sidewalls of the receiving cavity.

20. The fastening system of claim 15, wherein there are three blades arranged at angles of 120 degrees relative to each other, and three slots arranged at angles of 120 degrees relative to each other.

21. The fastening system of claim 15, wherein each blade has two surfaces and two protrusions located thereon, and each slot has two overhangs.

22. A blank for the manufacture of the fastener of claim 1, comprising
(a) a head having a centre and a surface defining a transverse axis, and
(b) an opposing shank end oriented perpendicularly to the head, said head comprising a cavity to receive the driving end of a driver tool bit therein; and
(c) at least one leaf on the surface of the head, the leaf being further defined by a hinge extending perpendicularly to the transverse axis.

23. A method of manufacturing the fastener of claim 1, comprising the steps of:
a. forming from a metal or alloy a blank comprising:
a head having a centre and a surface defining a transverse axis;
an opposing shank end oriented perpendicularly to the head, said head comprising a cavity to receive the driving end of a driver tool bit therein; and
at least one leaf on the surface of the head, the leaf being further defined by a hinge extending perpendicularly to the transverse axis; and
b. stamping the head so that the leaf folds at the hinge along the transverse axis towards the centre of the head.
